(19) United States
(12) Patent Application Publication
(10) Pub. No.: US 2003/0119034 A1
(43) Pub. Date: Jun. 26, 2003

(54) Biochip including carbon nanotubes and method for sample separation using the same

(76) Inventors: Seong-ho Kang, Gyeonggi-do (KR); Yukeun Eugene Pak, Gyeonggi-do (KR); Won-bong Choi, Gyeonggi-do (KR)

Correspondence Address: CANTOR COLBURN LLP, 55 Griffin South Road, Bloomfield, CT, 06002, US

(21) Appl. No.: 10/255,198

(22) Filed: Sep. 25, 2002

(30) Foreign Application Priority Data
Dec. 20, 2001 (KR) 2001-81780

(51) Int. Cl. C12Q 1/68

(52) U.S. Cl. 435/6

(57) ABSTRACT

A biochip and a method for separating a target substance contained in a sample on the biochip are disclosed. The biochip comprises a substrate, a sample loading portion disposed on the substrate, a channel in fluid communication with the sample loading portion, and carbon nanotubes arrayed in intervals in the channel. The sample is loaded on the sample loading portion and flowed through the channel. The target substance is selectively separated from the sample according to the interval sizes between the carbon nanotubes. According to the present invention, various substances in a sample may be easily separated or filtered by flowing through the channel having the carbon nanotubes, and thus, sample contamination and/or experimental error are reduced.

### BACKGROUND OF INVENTION

1. Field of Invention

The present invention relates to a method of separating and/or filtering a target substance from a sample, more particularly, to a biochip having a sample separation function and a method of separating and/or filtering a target substance from a sample using a biochip.

2. Description of Related Art

Biochips are a new technology that combines such technologies as biotechnology, nanotechnology and MEMS (Micro Electro Mechanical System) technology. Array-type DNA chips and protein chips are currently in the early commercialization phase. For instance, the concept of Lab-on-a-chip (LOC), i.e., DNA-LOC and protein-LOC, has been introduced. The DNA-LOC and protein-LOC techniques integrate several functions such as sample pre-treatment, derivatization, separation, detection, analysis, etc., in one chip. Various samples such as a natural materials, drugs, food, medicine, etc. as well as biological samples such as blood, urine, cells, saliva, etc., can thus be directly applied to such biochips. An example of the Lab-on-a-chip is disclosed in “Good-bye Test Tubes, Hello Labs-on-a-Chip” Stuart F. Brown, Fortune, Oct. 11, 1999.

But, if an undesirable substance exists in a sample, it can be difficult to obtain reliable data. For example, the hemoglobin in a red blood cell may inhibit a PCR (Polymerase Chain Reaction) experiment involving a sample containing red blood cells. See, McCusker, J., Dawson, M. T., Noone, D., Gannon, F., Smith, T. Nucleic Acids Res. 20, p 6747, 1992. Generally, the undesirable substance is eliminated by pre-treating the sample by centrifugation, by using a membrane (which can be made of various substances and has pores with various sizes, such as, for example, glass fiber), or by various kinds of filtration devices. But, there are still many problems in directly applying pre-treatment technology to a micro-channel or chamber of an LOC.

Recently, a method of filtering and isolating cells from blood has been developed. An example is disclosed in Po Ki Yuen et al., Genome research, 11, p 405-412, 2001/Peter Wilding et al., Analytical Biochemistry 257, p 95-100, 1998, in which a filter is disposed in a chip using MEMS technology. Another method of separating a DNA having a desired size is disclosed in Oligica Bakalin et al., Anal. Chem., in press, 2001. It is, however, difficult to make uniform spaces with nanometer size in a chip using these methods.

Carbon nanotubes are now a major component in miniaturized cathode-ray tubes (See: Nature 414, p 142-144, Nov. 8, 2001), and are applied to diverse technical fields. For example, nanometer-scale microscopy probes using carbon based nanotubes are disclosed in U.S. Pat. No. 6,159,742 entitled “NANOMETER-SCALE MICROSCOPY PROBES” to Charles M. Lieber, Stanislaus S. Wong, Adam T. Woolley, Ernesto Joselevich. Another example is disclosed in U.S. Pat. No. 6,139,919 entitled “METALLIC NANOSCALE FIBERS FROM STABLE IODINE-DOPED CARBON NANOTUBES” issued in 2000 to Eklund etc., in which stable iodine-doped carbon nanotubes or metallic nanometer-scale fibers are made by doping carbon nanotubes with iodine. Further examples are disclosed in U.S. Pat. No. 5,866,434 entitled “GRAPHITIC NANOTUBES IN LUMINESCEN ASSAYS” issued in 1999 to Richard J. Massey et al., in which electrochemiluminescent ruthenium complexes are made using functional group biomolecule-modified nanotubes.

However, a need still exists for separating or filtering a target substance from a sample disposed on a biochip using carbon nanotubes.

### SUMMARY OF THE INVENTION

Therefore, the present invention provides a biochip capable of separating or filtering a target substance from a sample containing various substances, so as to reduce sample contamination or experimental error.

Further, the present invention provides a method of separating or filtering a target substance from a sample containing various substances on a biochip to prevent sample contamination or experimental error.

Still further, the present invention provides a method of separating or filtering a target substance from a sample containing various substances by employing a plurality of carbon nanotubes disposed in an array-type channel wherein adjacent carbon nanotubes have various interval sizes.

In one aspect of the present invention, a biochip comprises a substrate, the substrate comprising a sample loading portion; a channel formed in the substrate and being in fluid communication with the sample loading portion; and a plurality of carbon nanotubes arrayed in predetermined intervals in the channel.

In another aspect of the present invention, a method of separating or filtering a target substance from a sample on a biochip comprises loading a sample on a sample loading portion of a substrate of a biochip; flowing the sample through a channel of the substrate, wherein the channel comprises a plurality of carbon nanotubes arrayed with predetermined intervals; and separating the target substance from the sample based on sizes of the intervals between the carbon nanotubes.

In still another aspect of the present invention, a method of separating a target substance from a sample containing various substances, comprises disposing a plurality of carbon nanotubes in a channel of a substrate, wherein the carbon nanotubes are arrayed in intervals; loading a sample comprising a target substance on the channel; flowing the sample through the channel, and separating the target substance.

In further aspect of the present invention, a method of making a biochip comprises disposing a metal layer on a substrate; oxidizing the metal layer to form a channel; and disposing a plurality of carbon nanotubes in the channel, such that the plurality of carbon nanotubes are arrayed in intervals.

### BRIEF DESCRIPTION OF THE DRAWINGS

The above objects and advantages of the present invention will become more apparent by detailed description on the preferred embodiments thereof with reference to the attached drawings in which:

FIGS. 1A-1D are schematic cross sectional views illustrating a process for preparing carbon nanotubes on a substrate, according to one embodiment of the present invention.

FIG. 2 is a schematic view illustrating a method of separating or filtering biological samples using carbon nanotubes, according to one embodiment of the present invention.

FIG. 3 is a top view of carbon nanotube filters disposed in channels or chambers of various shapes, according to one embodiment of the present invention.

FIG. 4 is a schematic view illustrating a process of separating white blood cells from a whole blood using multiple carbon nanotubes arrayed with various intervals in channels, according to another embodiment of the present invention.

### DETAILED DESCRIPTION OF THE INVENTION

According to preferred embodiments of the present invention, various substances contained in a sample can be separated or filtered while passing through channels or chambers, each of which includes a plurality of carbon nanotubes. For example, the substances can be separated based on the various sizes of the intervals between adjacent carbon nanotubes.

A biochip according to an embodiment of the present invention comprises a substrate having a sample loading portion and a channel. The channel is in fluid communication with the sample loading portion, and comprises a plurality of carbon nanotubes arrayed with predetermined intervals.

The substrate may be made from various materials such as silicon, melted silica, glass, and plastic. The channels or chambers may be made by disposing a metal layer on the substrate, and oxidizing the metal layer. As the metal layer, any metal generally used in the art, such as aluminum, can be used.

Preferably, the channel of the biochip includes a plurality of unit channels.

The intervals between the carbon nanotubes in the biochip can be varied depending on a diameter of a target substance to be separated. Preferably, the size of the intervals can be several nanometers to several hundred micrometers.

Each of the channels or chambers has a length suitable to accommodate a predetermined number of carbon nanotubes in array form with various intervals between the carbon nanotubes. Preferably, the length of the channel can be several nanometers to several hundreds millimeters.

A method of separating a target substance from a sample on a biochip according to another embodiment of the present invention comprises loading the sample, flowing the sample, and separating the target substance from the sample.

Loading the sample can be achieved by loading the sample on the sample loading portion located on the substrate of the biochip.

After loading on the sample loading portion, the sample is made to flow through the channel by a force such as an electric field, a pressure, a vacuum, an electromagnetic field, or a centrifugation force. Preferably, the flowing the sample comprises opening and closing of the channels, for instance, opening predetermined unit channels extending in a sample flow direction and closing the remaining unit channel(s). In this case, the sample is flowed through the opened unit channel(s).

Thus, the target substance included in the sample can be separated according to the sizes of the intervals between the carbon nanotubes.

Preferably, the target substance includes biological molecules, and more preferably, the target substance comprises one of cells, nucleic acids, DNAs, proteins, peptides, polysaccharides, hormones, lipids, carbohydrates, and receptors.

A method for separating a target substance contained in a sample, according to further embodiment of the present invention, comprises disposing a plurality of carbon nanotubes in an array-type arrangement in a channel such that there are various sizes of intervals between adjacent carbon nanotubes, loading a sample into the channel, flowing the sample through the channel, and separating the target substance.

The invention is further described by the following examples. The examples are only for the purposes of illustration. It should be understood that the invention is not limited to the specific details of the examples.

### EXAMPLE 1

### Preparation of Carbon Nanotubes

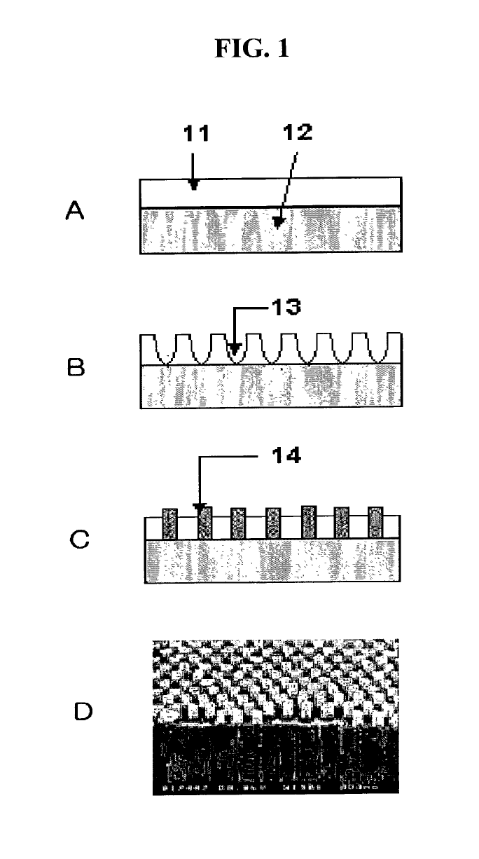

FIG. 1 shows a process of disposing carbon nanotubes on a substrate of a biochip.

Referring to FIG. 1, channels or chambers each having a length of nanometers to millimeters are prepared on a chip base substrate. The substrate may be made of various substances such as silicon, melted silica, glass, and plastic. A plurality of carbon nanotubes each having a length of nanometers to micrometers are prepared and arrayed in predetermined intervals in the channels or chambers.

For instance, the Al layer (11) was deposited on the substrate (12) as shown in FIG. 1A. Then, as shown in FIG. 1B, the Al layer (11) was oxidized using anodic aluminum oxidation to form channels or chambers (13) each having a length of tens of nanometers to hundreds of millimeters. Then, as shown in FIG. 1C, gases such as C₂H₂, and CH₄ are injected to array the carbon nanotubes (14).

The diameters of the carbon nanotubes and the sizes of the intervals between them may be controlled by the voltage and oxidizer used in the anodic aluminum oxidation.

FIG. 1D is the photograph of the carbon nanotubes formed horizontally.

### EXAMPLE 2

### Separating or Filtering Biological Samples Using Carbon Nanotubes Inside the Channels

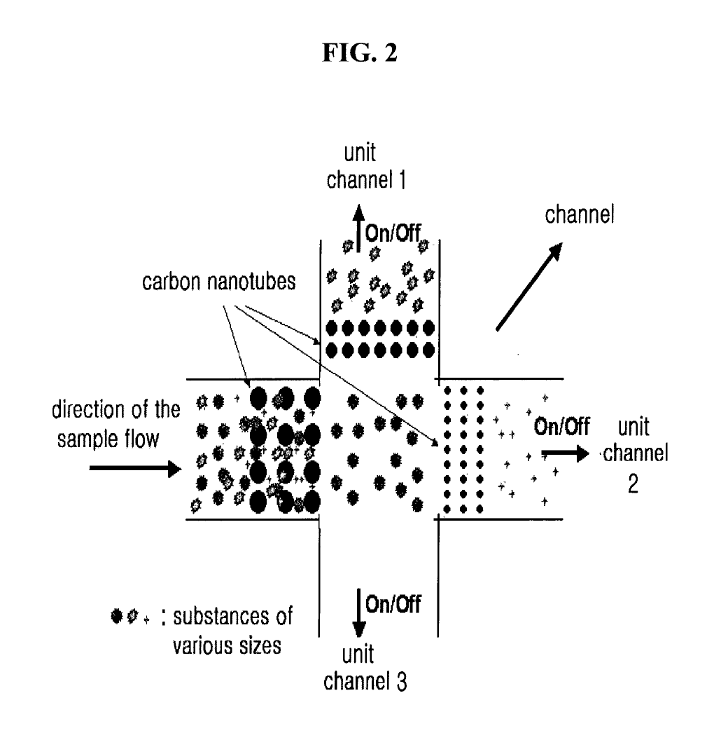

FIG. 2 illustrates one or more channels or chambers, each including a plurality of carbon nanotubes arrayed in various intervals. The channels or chambers can be used not only as a filtration device but also as a separation device. A force such as an electric field, a pressure, a vacuum, an electromagnetic field, and a centrifugation force may be applied to the channel so as to flow a fluid sample containing various substances.

For instance, a sample having a target substance and other various substances is disposed on the carbon nanotubes (which have different sizes and array patterns in each unit channel). Then, the sample may be moved in one direction by opening one or more unit channels extending in a sample flow direction and closing the remaining unit channel(s). Alternatively, a switch valve can be used to open and close the channels. By repeated opening and closing of the unit channel(s), the substances in the sample may be moved to other unit channels having a desired channel size after filtering through one unit channel.

For example, when a unit channel 2 is smaller than unit channels 1 and 3, the substances having sizes bigger than the size of the unit channel 2 are collected in a central area of the unit channels 1, 2, and 3. The smaller substances pass through the unit channel 2 when unit channel 2 is opened and the other two unit channels 1 and 3 are closed. The collected substances in the central area, which are too large to pass through the carbon nanotubes in the unit channel 2, can then be moved to the unit channel 1 by closing the unit channels 2 and 3 and opening the unit channel 1.

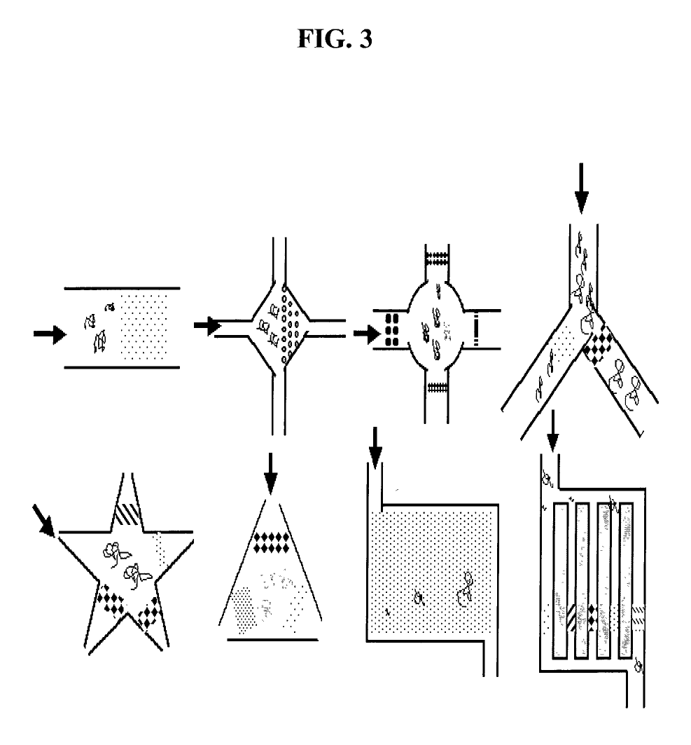

FIG. 3 shows channels or chambers of various shapes in which carbon nanotubes are formed. In the figure, arrows indicate the direction of sample injection.

Referring to FIG. 3, the channels or the chambers, which have various shapes, can be prepared, and the carbon nanotubes can be arrayed in appropriate positions of the channels or the chambers.

### EXAMPLE 3

### Separation of White Blood Cells from Whole Blood

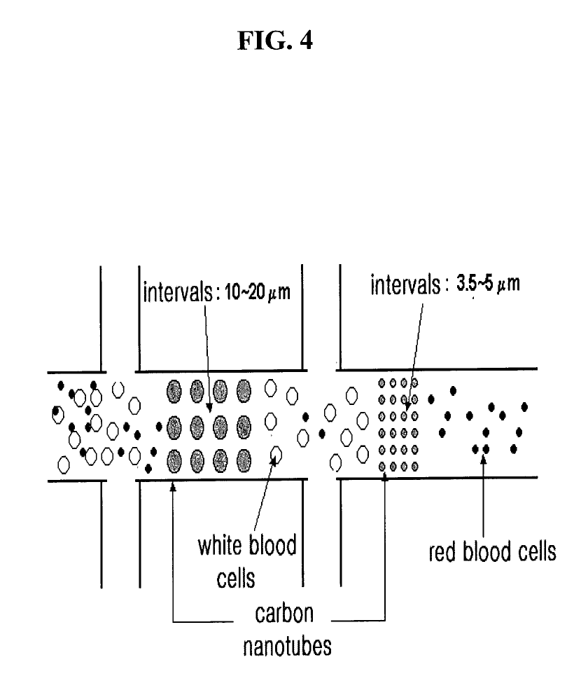

FIG. 4 shows a schematic diagram of a process for separating white blood cells from whole blood in the channels of a chip. The white blood cells can pass through the carbon nanotubes in a channel when the intervals between the carbon nanotubes are about 10 to about 20 micrometers. When the intervals between the carbon nanotubes are about 3.5 to about 5 micrometers, the white blood cells cannot pass through the carbon nanotubes, and only red blood cells can pass through and flow to downstream of the channels.

According to the present invention, a substance can be selectively separated or filtered from a sample having various substances based on its size. Further, the carbon nanotubes can be formed in channels or chambers of various geometrical shapes. Therefore, biochip having both high quality and various designs can be provided.

Further, in the present invention, carbon nanotubes of different sizes may be prepared for each of channels or chambers, thus comprehensive high-throughput carbon nanotube filters for separating or filtrating samples according to their sizes may be prepared. The carbon nanotubes can be applied to a Lab-on-a-Chip, thus sample contamination or experimental errors may be reduced.

While the invention has been described with respect to the specific embodiments, it should be recognized that various modifications and changes may be made by those skilled in the art to the invention which also fall within the scope of the invention as defined as the appended claims.

What is claimed is:

1. A biochip comprising;
a substrate, the substrate comprising a sample loading portion;
a channel formed in the substrate and being in fluid communication with the sample loading portion; and
a plurality of carbon nanotubes arrayed in intervals in the channel.

2. The biochip according to claim 1, wherein the channel comprises a plurality of unit channels.

3. The biochip according to claim 1, wherein the intervals between the carbon nanotubes are several nanometers to several hundred micrometers.

4. The biochip according to claim 1, wherein the channel has a length of several nanometers to several hundred millimeters.

5. The biochip according to claim 1, wherein the substrate comprises a material selected from the group consisting of silicon, melted silica, glass, and plastic.

6. The biochip according to claim 1, wherein the biochip comprises a Lab-on-a-chip.

7. A method of separating a target substance from a sample comprising:
loading a sample on a sample loading portion of a substrate of a biochip;
flowing the sample through a channel of the substrate, wherein the channel comprises a plurality of carbon nanotubes arrayed in intervals; and
separating the target substance from the sample.

8. The method according to claim 7, wherein the channel comprises a plurality of unit channels.

9. The method according to claim 8, wherein the flowing the sample through the channel comprises:
opening a unit channel extending in a sample flow direction to form an opened unit channel;
closing the remaining unit channels; and
flowing the sample through the opened unit channel.

10. The method according to claim 7, wherein the target substance comprises a biological molecule.

11. The method according to claim 10, wherein the biological molecule is selected from the group consisting of cells, nucleic acids, DNAs, proteins, peptides, polysaccharides, hormones, lipids, carbohydrates, and receptors.

12. The method according to claim 7, wherein the flowing the sample through the channel comprises applying a force selected from the group consisting of an electric field, a pressure, a vacuum, an electromagnetic field, and centrifugation.

13. A method of separating a target substance from a sample comprising;
disposing a plurality of carbon nanotubes in a channel of a substrate, wherein the carbon nanotubes are arrayed in intervals;
loading a sample in the channel;
flowing the sample through the channel; and
separating the target substance from the sample.

14. The method according to claim 13, wherein disposing a plurality of carbon nanotubes comprises:
forming a plurality of unit channels in the substrate; and
disposing the carbon nanotubes in the unit channels.

15. The method according to claim 14, wherein the flowing the sample through the channel comprises:
opening a predetermined unit channel extending in a sample flow direction;
closing the remaining unit channels; and
flowing the sample through the opened unit channel.

16. The method according to claim 13, wherein the target substance comprises a substance selected from the group consisting of cells, nucleic acids, DNAs, proteins, peptides, polysaccharides, hormones, lipids, carbohydrates, and receptors.

17. A method of making a biochip comprising:
disposing a metal layer on a substrate;
oxidizing the metal layer to form a channel; and
disposing a plurality of carbon nanotubes in the channel, such that the plurality of carbon nanotubes are arrayed in intervals.
